(19) United States
(12) Patent Application Publication
(10) Pub. No.: US 2024/0043354 A1
(43) Pub. Date: Feb. 8, 2024

(54) Producing Hydrocarbons From Carbohydrates Using Nuclear Energy

(71) Applicant: The Earthineering Company LLC, Altadena, CA (US)

(72) Inventor: Kevin Peter HICKERSON, Altadena, CA (US)

(73) Assignee: The Earthineering Company LLC, Altadena, CA (US)

(21) Appl. No.: 18/366,838

(22) Filed: Aug. 8, 2023

Related U.S. Application Data
(60) Provisional application No. 63/395,925, filed on Aug. 8, 2022.

(51) Int. Cl. C07C 1/04; G21D 9/00; C10L 1/06

(52) U.S. Cl. CPC C07C 1/0495; G21D 9/00; C10L 1/06; C10L 2270/04; C10L 2200/0492; C01B 32/05

(57) ABSTRACT

A system and method produce industrial hydrocarbons and other molecules in three coordinated reactors using nuclear process heat. A nuclear reactor generates heat via a nuclear process, and this heat is physically carried to one or both of the other reactors via a thermal medium. A gasification reactor uses the nuclear heat for a gasification process that converts biomass, e.g. cellulose or other polysaccharides, into gaseous hydrogen and carbon monoxide (i.e. syngas). The gasification process may be anaerobic, without requiring addition of purified or atmospheric oxygen or consumption of input biomass to provide the process heat. The syngas is carried to a Fischer-Tropsch reactor for conversion into desired output molecules, such as aviation or diesel fuel or carbon fibers, according to a selected chemical process.

### CROSS REFERENCE SECTION

This application claims the benefit of U.S. Provisional Application No. 63/395,925 filed on Aug. 8, 2022. That application is incorporated herein by reference in its entirety.

### BACKGROUND

The primary production of aviation fuel and other high-energy hydrocarbons continues to depend on exploitation of non-renewable oil and gas resources. In addition to the growing demand for fuel for the aviation industry, the demand for certified carbon neutral fuels is likely to dramatically increase in markets where government agencies and companies push for a lower fossil fuel carbon footprint. However, the demand for increased reliance on renewable sources of hydrocarbon products is only going to become more profound as the planet warms up. There is thus a need for a method of producing high-energy hydrocarbons that does not increase net carbon in the atmosphere.

Nuclear fission is the nuclear process when an atomic nucleus splits into two or more pieces. In certain heavy isotopes this also releases an enormous amount of energy. This is frequently accompanied also, by the release of one or more neutrons. Neutron induced fission only occurs when a neutron hits a nucleus first and causes it to undergo fission and split. This is often accompanied by the release of energy, but also one or more extra neutrons. When more neutrons come out than go in, this has the potential to create a nuclear chain reaction. By controlling the flux of neutrons, this chain reaction can be controlled too, releasing a steady stream of enormous amounts of energy. This is the fundamental source of power in a nuclear reactor.

The most common type of fission in a nuclear reactor is the fission of uranium-235, a slightly lighter isotope of uranium than the more common uranium-238. A typical example fission reaction is

²³⁵U+n→⁹²Kr+¹⁴¹Ba+3n+energy.

The exact split of the atom is random. Many so-called fission daughter nuclei are possible, but the average specific energy for uranium-235 fission is 83.2 terajoules per kilogram. This is 1.8 million times greater than the specific energy stored in jet fuel and similar liquid hydrocarbons and coal.

### SUMMARY OF DISCLOSED EMBODIMENTS

Disclosed embodiments synthesize hydrocarbons from biomass discards generated in massive quantities by other industry segments, such as farming. This is accomplished using three reactors, each with its own “control zone” (or simply “zone”). The “blue zone” provides a large quantity of heat using a nuclear reactor. The “green zone” uses this heat to convert biomass (primarily cellulose) into a mixture of hydrogen gas (H₂) and carbon monoxide (CO), commonly known as syngas. The “red zone” uses a Fischer-Tropsch process to convert the syngas into a variety of useful hydrocarbons, such as jet fuel, rocket fuel, gasoline and synthetic oils, basic chemicals such as ethylene, advanced carbon fibers, and an almost countless number of other potential carbon-based products.

Advantageously, embodiments are much better for the environment than existing process plants that remove sequestered carbon from the ground (in the form of fossil fuels) and emit it into the atmosphere, because embodiments instead recycle above-ground, low-quality biomass into high-energy density fuels rather than having it used for e.g. livestock feed. Unlike traditional biomass conversion plants that rely on hydrocarbon fuels to power their processes, disclosed embodiments leverage the low cost of energy-dense nuclear process heat. By using temperatures of over 900° C. (1652° F.) in the gasification reactor, thermal efficiency is greatly improved. Moreover, by using nuclear power as an external heat source, a larger syngas output per unit of biomass input is achieved than in conventional plants, because excess biomass input is not required to be burned to produce the heat of gasification.

Embodiments also may perform anaerobic gasification, or gasification that does not require oxygen to generate heat by burning the biomass input. Anaerobic gasification has other technical advantages over prior art implementations, e.g. it does not require purifying oxygen out of the air, or using air directly. Use of air exposes the syngas to gaseous nitrogen, which can also react with the biomass inputs to produce nitrogen containing compounds, for example, ammonia or amines. While these may have some value, extracting them requires additional equipment and processing before sending the syngas to the Fischer-Tropsch reactor, as they are not wanted or needed for the production of hydrocarbons. Further, when oxygen or air is used for heating, carbon dioxide is also created as a large component of the gasification output, and this must be removed before the Fischer-Tropsch process. In both these cases, multiple gases need to be processed and removed, adding complexity. By contrast, disclosed embodiments may eliminate this added complexity by only adding heat through a heat exchanger, without requiring extra gas processing.

Thus, a first embodiment is a system comprising a nuclear reactor for producing heat in a first medium; a heat exchanger, thermally coupled to the nuclear reactor, for exchanging heat between the first medium and a second medium; and a gasification reactor for receiving heat from the second medium to convert input biomass into output syngas.

In some embodiments, the nuclear reactor produces heat using tri-structural isotropic (TRISO) particle fuel.

In some embodiments, the first medium comprises helium and the second medium comprises helium, or a molten salt, or steam, or a liquid metal.

In some embodiments, the first medium has a temperature of up to 1600° C. and the second medium has a temperature of between 800° C. and 1300° C.

In some embodiments, the nuclear reactor further produces radioactive medical isotopes.

Some embodiments have a Fischer-Tropsch reactor receiving the syngas for converting the syngas into one or more selected hydrocarbons using a Fischer-Tropsch process.

In some embodiments, one or more of the selected hydrocarbons comprises aviation fuel or carbon fiber.

In some embodiments, the heat used in the Fischer-Tropsch process is received primarily from the received syngas.

In some embodiments, a temperature of the Fischer-Tropsch process does not exceed 800° C.

Another embodiment is a method comprising producing heat in a first medium using a nuclear reactor; exchanging heat between the first medium and a second medium using a heat exchanger thermally coupled to the nuclear reactor; and converting input biomass into output syngas using a gasification reactor that receives heat from the second medium.

In some embodiments, producing heat in the first medium comprises producing nuclear reactions inside tri-structural isotropic (TRISO) particle fuel.

In some embodiments, the first medium comprises helium and the second medium comprises helium, or a molten salt, or a liquid metal.

In some embodiments, exchanging heat comprises raising the second medium to a temperature of between 800° C. and 1300° C.

Some embodiments include producing radioactive medical isotopes using the nuclear reactor.

Some embodiments include converting the syngas into one or more selected hydrocarbons using a Fischer-Tropsch process in a Fischer-Tropsch reactor.

In some embodiments, one or more of the selected hydrocarbons comprises aviation fuel or carbon fiber.

In some embodiments, the heat used in the Fischer-Tropsch process is received primarily from the second medium and the received syngas.

In some embodiments, a temperature of the Fischer-Tropsch process does not exceed 800° C.

It is appreciated that the concepts, techniques, and structures disclosed herein may be embodied by a person having ordinary skill in the art in ways other than those summarized above. Therefore, the above summary of disclosed embodiments is and should be viewed as merely illustrative, not limiting.

### DESCRIPTION OF THE SEVERAL VIEWS OF THE DRAWINGS

The manner and process of making and using the disclosed embodiments may be appreciated by reference to the drawings, in which:

FIG. 1 schematically shows a system embodiment of the concepts, techniques, and structures disclosed herein, in a typical environment;

FIG. 2 schematically shows relevant components of the system, and their respective relationships;

FIG. 3 schematically shows relevant processes that are performed by the system, and their respective relationships; and

FIG. 4 is a flow diagram for a method embodiment of the concepts, techniques, and structures disclosed herein.

### DETAILED DESCRIPTION OF EMBODIMENTS

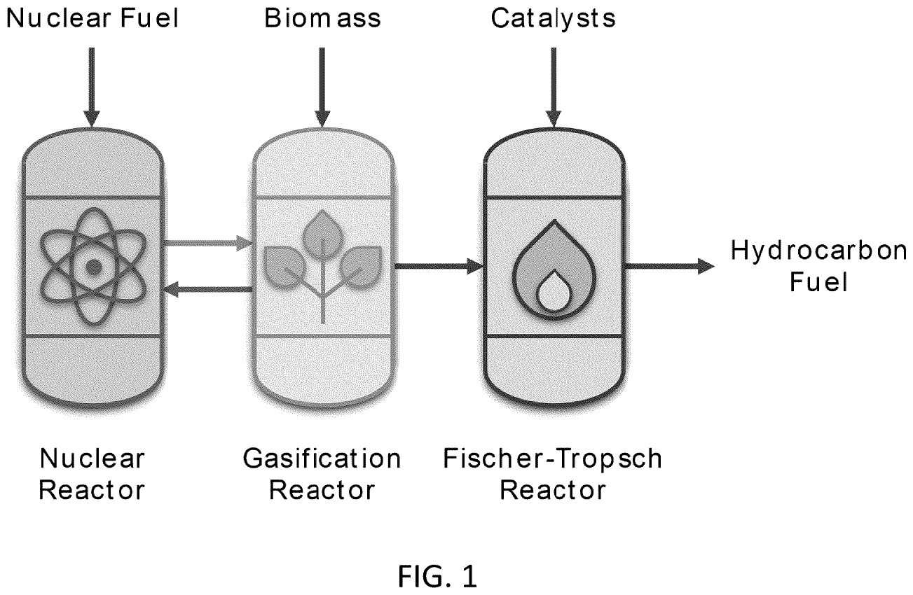

FIG. 1 schematically shows a system embodiment of the concepts, techniques, and structures disclosed herein, in a typical environment. The system has three different zones of operation.

The first or “Blue” Zone produces process heat using a nuclear reactor that provides nuclear fission. The nuclear reactor may be of a type known in the art, such as a fourth-generation, high-temperature, gas-cooled pebble bed reactor (PBR). Techniques for building and operating such reactors are known in the art. The relevant input to the Blue Zone is nuclear fuel. In some useful embodiments, the nuclear fuel “pebbles” comprise tri-structural isotropic (TRISO) particles. The outputs of the blue zone are high-temperature heat into the other two zones, optionally radioactive medical isotopes and other nuclear materials of interest, and spent fuel. For safety reasons, the highest temperatures inside the Blue Zone do not exceed a temperature of about 1600° C., including inside the reactor during a complete failure of every nuclear control system. This is well below the breakdown temperature for TRISO fuel particles, ensuring that they remain intact and physically cannot “melt down.”

The second or “Green” Zone implements a gasification process that converts biomass into syngas. As used herein, “syngas” means a gas composed primarily by weight of diatomic hydrogen (H₂) and carbon monoxide (CO). The inputs to the Green Zone are agricultural matter, forestry slash, or other raw, or pre-process biomass material containing mostly cellulose, and heat obtained from the Blue Zone. The outputs of the Green Zone are syngas and un-proces sable waste products such as sulfur, rocks, dirt, salts, and heavy metals. Techniques for building and operating gasification reactors are known in the art.

No unnaturally occurring radiological material is transferred from the Blue Zone to Green Zone. This means that the Green Zone can be considered a nuclear-free area. Nevertheless, the Green Zone is in close, physical proximity to the Blue Zone (e.g. within 100 yards) to maximize the efficiency of physical transport of the heat-bearing medium between the zones and to minimize heat loss to the environment. The highest temperatures inside the Green Zone do not exceed 1300° C., except possibly in processes having a small and controlled energy volume such as catalyst recycling.

The third or “Red” Zone implements a Fischer-Tropsch process that converts the syngas into desired hydrocarbons. The Red Zone contains materials with the highest flammability and potentially even explosive risk, so minimizing potential fire damage to the Blue and Green Zones is critical. Temperatures in the Red Zone do not exceed 800° C., including inside reactors. The inputs to the Red Zone are syngas, process heat below 800° C. from the Blue Zone or held within the syngas from the Green Zone, additives (e.g. for jet fuel or other output products), and Fischer-Tropsch catalysts (e.g. various transition metals, described below in more detail). The outputs of the Red Zone are hydrocarbon fuel products including Jet A-1 or Jet A, diesel fuel, gasoline, and ethylene, as well as synthetic oils, carbon fibers and polymers, along with fouled Fischer-Tropsch catalysts. Techniques for building and operating Fischer-Tropsch reactors are known in the art.

The Red Zone is in close, physical proximity to the Blue Zone and the Green Zone (e.g. within hundreds of yards). It does not need to be as close as the Green Zone is to the Blue Zone, as the maximum temperatures in the Red Zone are much lower than in the Green Zone so heat loss from transportation of the second medium is not as important an issue. However, syngas is very diffuse, and thus does not hold a great deal of heat content, so pipes carrying hot syngas from the Green Zone to the Red Zone are often large, and therefore should be as short as possible while avoiding potential fire and explosion damage to the Green Zone.

Other advantages arise due to the close, physical proximity between the zones, and the use of TRISO nuclear fuel. In particular, due to the nature of TRISO fuel pebbles, the Nuclear Regulatory Commission has determined that a radiological release of nuclear contaminants in a TRISO reactor due to an accident would require a radiological exclusion zone of only 400 yards. As noted above, temperatures inside the Blue zone prevent any radiation leakage from a meltdown of TRISO fuel. Nevertheless, it is desirable as a backup precaution for any potential exclusion zone to lie entirely within the boundaries of a process plant that includes all three Zones, i.e. a location already fully under the control of the plant's owner-operator. Thus, there is no radiological risk to the general public from the operation of the plant.

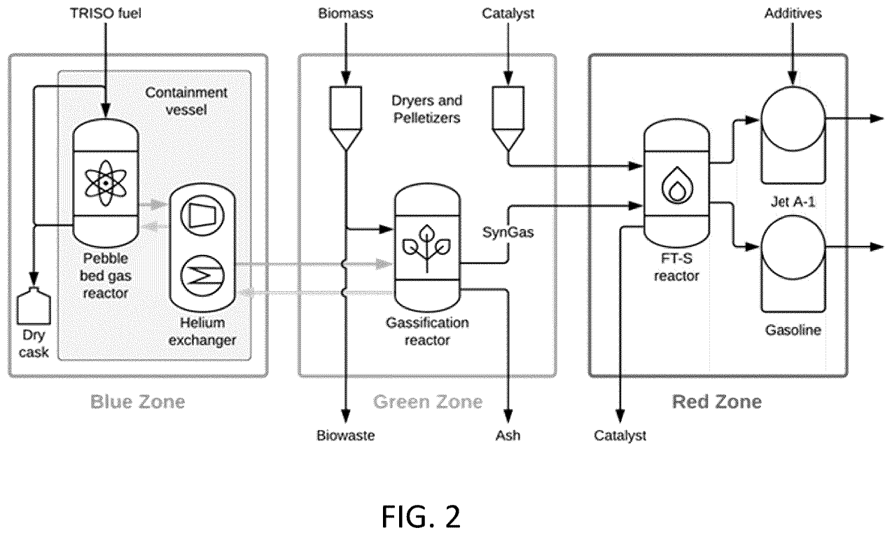

FIG. 2 schematically shows relevant physical components of the Blue, Green, and Red Zones shown in FIG. 1, and their respective relationships.

The Blue Zone is used to generate nuclear process heat for use in the Green and Red Zones. Thus, the Blue Zone includes a nuclear reactor, which may be a pebble bed gas reactor (PBR). The reactor receives nuclear fuel and subjects it to nuclear reactions to produce nuclear process heat. The reactor output is spent fuel, which may be recycled as shown, or stored in dry casks as known in the art.

The reactor may use fuel that will not melt, such as TRISO fuel particles. In some embodiments, the TRISO particle is made up of a uranium, carbon, and oxygen fuel kernel. Some embodiments may also include thorium or plutonium as a fissile material. The kernel is encapsulated by three layers of carbon- and ceramic-based materials that prevent the release of radioactive fission products. The particles are incredibly small (about the size of a poppy seed) and very robust. They can be fabricated into cylindrical pellets or billiard ball-sized “pebbles” for use in either high temperature gas or molten salt-cooled reactors.

TRISO fuels are structurally more resistant to neutron irradiation, corrosion, oxidation and high temperatures (the factors that most impact fuel performance) than traditional reactor fuels. They are, by design, self-cooling in the case of loss of reactor control, using natural convection until control rod or coolant flow can be reacquired. Each particle acts as its own containment system thanks to its triple-coated layers. This allows them to retain fission products under all reactor conditions.

TRISO pebbles are safe to hold before they have been placed in the reactor. After they have passed through the reactor, they contain fission products and will spontaneously emit gamma radiation. These pebbles cannot contaminate or make material outside the graphite shell become radioactive one they are not undergoing fission chain reaction. However, after they undergo fission, they will continue to create heat and radiation for years.

The heat from the nuclear reaction process is stored in a first medium, which may be helium gas (He) which is chemically and radiologically inert. Helium gas is heated by the radioactive TRISO pebbles to temperatures that are not possible with steam, e.g. up to 1600° C. This high temperature helium gas is sent to a specialized heat exchanger used to transfer heat into a second medium for use by chemical processes outside the reactor containment vessel (i.e. in the Green Zone and Red Zone). The second medium may be helium, molten salt, liquid metal, or steam, and is raised to a temperature of between 800° C. and 1300° C., while the cooler first medium is sent back to the nuclear reactor to be reheated.

To extract heat from the reactor, and pass it to a secondary working fluid, there must be a large and dedicated helium pumping and heat exchanger system, called an Intermediate Heat exchanger (IHX). The IHX is connected directly to the main nuclear reactor vessel via a hot helium duct and a coaxial cold return duct. This apparatus is completely sealed inside a reinforced concrete radiological containment vessel. This will keep the IHX safe, even from extreme events, such as earthquakes and passenger jet collisions.

The purpose of this intermediate exchanger is to transfer heat from gas that has been in direct physical contact with the core, to a secondary working fluid that can enter and exit containment, but is never in direct contact with any radiological materials inside the core itself. This way, there is no pathway to allow radiological material out of containment, but allows for usable heat to flow in and out through the secondary fluid. This is important because even though the radioactive material that is in the core is very controlled in these advanced reactors due to advanced design and operational purification systems, there is the possibility that some material might mix with the reactor coolant gas. The most likely of this material is carbon-14, a naturally occurring radioisotope that is produced when neutrons interact with the graphite moderators and the TRISO fuel in the core. This reactor coolant gas cannot leave the Blue Zone or the containment vessel.

Helium itself cannot become radioactive by passing through a reactor except for an extremely rare helium-6 state which has a half-life of less than 1 second. But air, argon, water, carbon dioxide, and graphite dust can become radioactive, and small quantities of radioactive particles from fission of the TRISO fuel may escape. These are filtered out as a normal part of plant operations. The IHX solves the problem of radioactivity leakage by being inside the containment vessel, while still transferring heat efficiently to another working fluid that has no new radioactive isotopes created in the heat exchange.

The Green Zone is used for gasification of biomass. It stores input biomass to be used, from which unusable biowaste is extracted. The remaining useful biomass is provided to the gasification reactor. As indicated above, the gasification reactor receives heat from the second medium to convert the useful biomass into output syngas. The portions of the biomass that are not converted to syngas are removed from the gasification reactor as ash. The Green Zone also may store fuel additives and catalysts used in the Red Zone, to provide physical distance between the catalysts and the Red Zone in case of a fire.

Gasification is the process that converts biomass materials at high temperatures, without combustion, into carbon monoxide (CO), hydrogen (H₂), nitrogen (N₂), carbon dioxide (CO₂), and other residual gases. The dominant reaction is the use of cellulose and similar natural polysaccharides as the biomass feedstock, but this requires a lot of energy in the form of heat to be pumped into the gasification reactor.

Traditional gasification of cellulose for the creation of biofuels introduced oxygen into the gasification reactors to combust a fraction of the biomass, in order to drive the heat needed for the rest of the process to occur. By contrast with the prior art, in disclosed embodiments no biomass is burned and no atmospheric oxygen needs to be introduced. Instead, the energy needed to gasify the biomass is provided by the secondary heating fluid (helium, steam, or molten salt) received from the Blue Zone.

Gasification typically takes place in stages and at different temperatures. A first process is dehydration of the biomass, which occurs at 100° C. Steam produced at this step can be kept in the reactor gas or removed. A second step is pyrolysis or devolatilization, which occurs at 200-300° C. This process releases volatile chemicals such as methane, hydrogen, and others from the feedstock, which in this stage becomes char. This process is heavily dependent on the feedstock chemistry.

A third process is cellulose combustion via the chemical reaction

(C₆H₁₀O₅)ₙ+6n O₂→5n H₂O+6n CO₂.

This process occurs if there is oxygen present after the volatiles begin to release from the biomass char. Prior art gasifiers intentionally add excess oxygen to this process to heat to the reaction. By contrast, disclosed embodiments do not require the addition of excess oxygen, or burning other biomass to produce the necessary heat, which instead comes from the Blue Zone.

A fourth process is cellulose hydrolysis via the chemical reaction

(C₆H₁₀O₅)ₙ+n H₂O→6n C₆H₁₂O₆.

In this process, the remaining cellulose chains are broken down and a water molecule (from the combustion process) is added to each polymer unit to create high temperature glucose.

A fifth and final process on the main chemical pathway is glucose gasification, where steam reacts at 700-800° C. with the glucose char to create hydrogen and carbon monoxide, or syngas, via the chemical reaction

C₆H₁₂O₆→6 H₂+6 CO.

As noted, volatiles are produced in the second, pyrolytic process. Steam methane reforming is an additional endothermic process that can be used in the reactor to enhance the production of hydrogen content of syngas from steam and methane (CH₄) via the chemical reaction

CH₄+H₂O→3 H₂+CO.

Autothermal reforming can also be used to consume excess methane, as well as provide an exothermic boost up to 900°-1100° C. The process can use excess or even captured carbon dioxide (CO₂) via the reaction

2 CH₄+O₂+CO₂→3 H₂+3 CO+H₂O,

or steam for boosting the output hydrogen fraction via the reaction

4 CH₄+O₂+2 H₂O→10 H₂+4 CO.

The Red Zone converts syngas into desired hydrocarbon products via one or more selected Fischer-Tropsch reactions. Once syngas is created in the Green Zone, it can be transferred by insulated gas pipe to the Red Zone for the creation of hydrocarbons. This is accomplished primarily by using the Fischer-Tropsch synthesis global reaction

(2n+1)H₂+n CO→Cₙ H₍₂ₙ₊₂₎+n H₂O.

Catalysts are essential in the Fischer-Tropsch process. Commonly employed catalysts include iron (Fe), cobalt (Co), ruthenium (Ru), and nickel (Ni). Iron is low cost, has a high water-gas-shift activity, and is preferable for mixtures of syngas having a relatively low H₂/CO ratio. Iron catalysts may contain promoters, such as potassium and copper, and high surface area binders/supporters such as silica, alumina, or both. Cobalt is an active catalyst, and is more effective at low temperatures. At higher temperatures, cobalt produces more methane output. Ruthenium is the most active catalyst, but is very expensive. It works at the lowest reaction temperatures, and it produces the highest molecular weight hydrocarbons, without the need for promoters. The use of nickel results in the greatest amount of methane formation.

A key variable in producing a differential product distribution is the operating temperature of the Fischer-Tropsch reaction. A higher temperature produces a higher gasoline/diesel ratio (2/1), while a low temperature produces the reverse ratio. This is independent of the type of catalyst (either Fe or Co). Higher temperatures produce lower carbon number products, as well as more hydrogenated products. There is also more branching and an increase of secondary products such as ketones and aromatics.

Two types of Fischer-Tropsch reactors that may be used are a fixed fluidized bed reactor that exhibits a high throughput at high temperatures, and a slurry-bed reactor that has better temperature controls and higher conversion rates.

A general rule of thumb is that complex hydrocarbon fuels deteriorate over time while sitting in storage. Disclosed embodiments are designed to ensure that finished products spend as little time at the production site (Red Zone) as possible. Even so, containment and handling of volatile products requires special attention and processes. Storage tanks will meet Federal and State safety regulations as well as standards set by ASME, OSHA, BPVC and others. Product handling procedures should meet all OSHA and environmental requirements. This includes ensuring products do not enter the local groundwater, above and beyond EPA requirements.

As shown in FIG. 2, the output products of the Fischer-Tropsch process may include hydrocarbon fuels that require additives. These additives may be received and stored on-site (i.e. in the Green Zone) until they are needed, or may be received in the Red Zone and mixed with the output products via just-in-time manufacturing principles. Other output products of the process may include spent catalyst, as shown. These processes are described in more detail below.

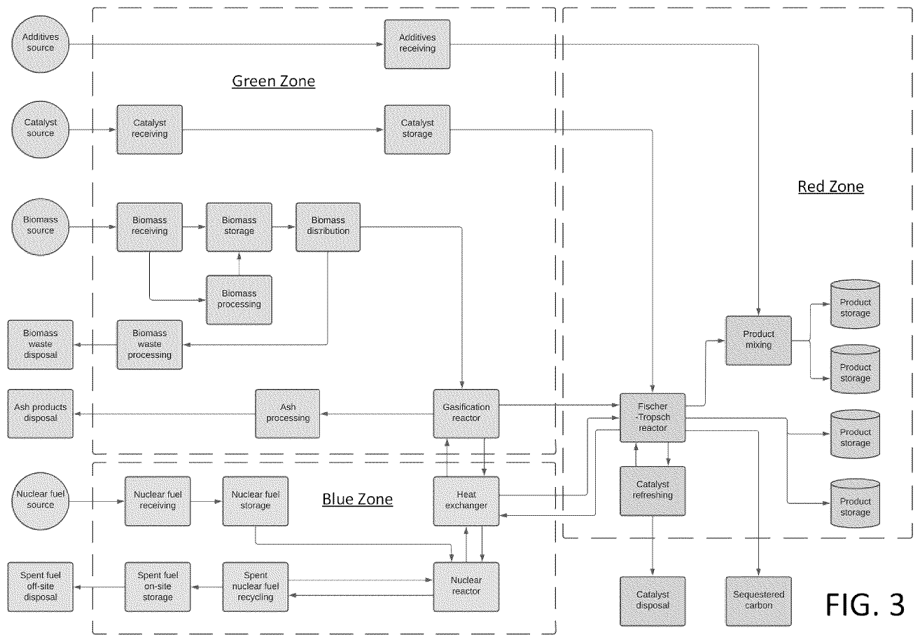

FIG. 3 schematically shows relevant processes that are performed to operate the system, e.g. as a nuclear and chemical process plant. Most of these processes were described above in connection with FIG. 2, and many are conventional, so they are only summarized here.

An external nuclear fuel source provides fuel (e.g. TRISO pebbles), which are received in the Blue Zone in a nuclear fuel receiving area, then moved internally by the owner-operator into a nuclear fuel storage area. Once they are needed, they are moved into the nuclear reactor, where they are consumed. From time to time, spent nuclear fuel is then sent to a recycling area, where quality control is performed to include evaluating whether each pebble is suitable for reintroduction into the reactor. Pebbles that still have a useful lifetime are sent back to the reactor, as indicated, while fully spent fuel is moved to on-site storage in dry casks within the Blue Zone. Excess spent fuel may be further removed from the Blue Zone to an external off-site disposal or storage location. As noted above, nuclear process heat is conveyed by a first medium (e.g. helium) to a heat exchanger, where it is transferred to a second medium (e.g. helium, molten salt, or liquid metal) for transport outside the Blue Zone.

Notably, all individuals who work in the Blue Zone may undergo heightened training in nuclear material handling and security background checks that are not necessary for workers in the Green and Red Zones, and the Blue Zone perimeter may have high security exclusion measures (e.g. barbed wire fences and armed guards). However, nuclear process heat crosses the perimeter into the Green and Red Zones at defined locations, in non-radioactive mediums, with only basic safety precautions (e.g. against persons coming into contact with hot pipes). In particular, nuclear process heat could be sent to on-site steam turbines (e.g. in the Green Zone) to produce electrical power consumed in the three Zones, or off-site, thereby resulting in a chemical process plant that is a net energy producer rather than a net energy consumer.

An external biomass source, e.g. a farm, provides biomass which is received in the Green Zone in a biomass receiving area. Some biomass is moved to biomass storage areas (e.g. silos), while other biomass is pre-processed by drying and pelletizing before moving to the storage areas. Once needed, biomass is removed from storage and separated. Cellulose is forwarded to the gasification reactor, while biomass waste is processed for off-site disposal. As noted above, the gasification reactor in the Green Zone is heated primarily by nuclear process heat which has been moved into a non-radioactive (second) medium. The primary product of gasification, hot syngas, is transported by pipe to the Red Zone, while the remaining ash is processed and sent for off-site disposal.

In addition to gasification of cellulose, the Green Zone may also receive additives (e.g. fuel additives) and catalysts for use in the Red Zone. Additives have relatively short shelf-lives, and therefore may or may not be stored in the Green Zone, but may be received there (rather than in the Red Zone) to facilitate interaction with external shippers in a location having minimal risk of fire or explosion. They then may be securely transported on-site between the Green and Red Zones by the owner-operator of the plant. Similarly, catalysts may be received in the Green Zone. Unlike fuel additives, however, metal catalysts have a long shelf-life and may be stored in the Green Zone until they are needed in the Red Zone.

The Red Zone does not receive any inputs that are external to the system. Rather, the Red Zone Fischer-Tropsch reactor receives nuclear process heat from the Blue Zone, and hot syngas and catalysts from the Green Zone. The direct hydrocarbon output products of the Fischer-Tropsch reactor may be stored in the Red Zone as shown, or sent to an additive mixing area where they are mixed with additives received from the Green Zone prior to storage. Direct reaction products also may include sequestered carbon, which is sent off-site for disposal. From time to time, catalysts used in the Fischer-Tropsch reactor are removed and refreshed at a catalyst refreshing area. Refreshed catalysts are returned to use in the Fischer-Tropsch reactor, while fully spent catalysts are sent off-site for disposal.

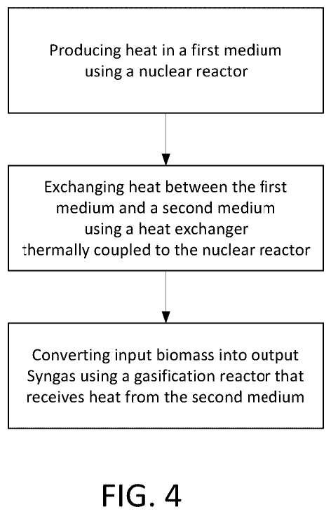

FIG. 4 is a flow diagram for a method embodiment of the concepts, techniques, and structures disclosed herein. The method of FIG. 4 may be implemented using a system described above in connection with FIGS. 1, 2, and 3. The method begins with a nuclear reaction process producing heat in a first medium using a nuclear reactor. The method includes a heat exchanging process exchanging heat between the first medium and a second medium using a heat exchanger thermally coupled to the nuclear reactor. The method then has a gasification process converting input biomass into output syngas using a gasification reactor that receives heat from the second medium.

It is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the several purposes of the disclosed subject matter.

In the foregoing detailed description, various features of embodiments are grouped together in one or more individual embodiments for the purpose of streamlining the disclosure. This method of disclosure is not to be interpreted as reflecting an intention that the claims require more features than are expressly recited therein. Rather, inventive aspects may lie in less than all features of each disclosed embodiment.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

As used herein, “including” means including without limitation. As used herein, the terms “a” and “an”, when modifying a noun, do not imply that only one of the noun exists. As used herein, unless the context clearly indicates otherwise, “or” means and/or. For example, A or B is true if A is true, or B is true, or both A and B are true. As used herein, “for example”, “for instance”, “e.g.”, and “such as” refer to non-limiting examples that are not exclusive examples. The word “consists” (and variants thereof) are to be give the same meaning as the word “comprises” or “includes” (or variants thereof).

Various embodiments of the concepts, systems, devices, structures and techniques sought to be protected are described herein with reference to the related drawings. Alternative embodiments can be devised without departing from the scope of the concepts, systems, devices, structures and techniques described herein. It is noted that various connections and positional relationships (e.g., over, below, adjacent, etc.) are set forth between elements in the following description and in the drawings. These connections and/or positional relationships, unless specified otherwise, can be direct or indirect, and the described concepts, systems, devices, structures and techniques are not intended to be limiting in this respect. Accordingly, a coupling of entities can refer to either a direct or an indirect coupling, and a positional relationship between entities can be a direct or indirect positional relationship.

As an example of an indirect positional relationship, references in the present description to forming layer “A” over layer “B” include situations in which one or more intermediate layers (e.g., layer “C”) is between layer “A” and layer “B” as long as the relevant characteristics and functionalities of layer “A” and layer “B” are not substantially changed by the intermediate layer(s).The following definitions and abbreviations are to be used for the interpretation of the specification. As used herein, the terms “comprises,” “comprising, “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance, or illustration. Any embodiment or design described herein as “exemplary” is not necessarily to be construed as preferred or advantageous over other embodiments or designs. The terms “one or more” and “one or more” are understood to include any integer number greater than or equal to one, i.e. one, two, three, four, etc. The terms “a plurality” are understood to include any integer number greater than or equal to two, i.e. two, three, four, five, etc. The term “connection” can include an indirect “connection” and a direct “connection.”

References in the specification to “one embodiment, “an embodiment,” “an example embodiment,” etc., indicate that the embodiment described can include a particular feature, structure, or characteristic, but every embodiment can include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

For purposes of the description hereinafter, the terms “upper,” “lower,” “right,” “left,” “vertical,” “horizontal, “top,” “bottom,” and derivatives thereof shall relate to the described structures and methods, as oriented in the drawing figures. The terms “overlying,” “atop,” “on top, “positioned on” or “positioned atop” mean that a first element, such as a first structure, is present on a second element, such as a second structure, where intervening elements such as an interface structure can be present between the first element and the second element. The term “direct contact” means that a first element, such as a first structure, and a second element, such as a second structure, are connected without any intermediary elements.

Use of ordinal terms such as “first,” “second,” “third,” etc., in the specification to modify an element does not by itself connote any priority, precedence, or order of one element over another or the temporal order in which acts of a method are performed, but are used merely as labels to distinguish one element having a certain name from another element having a same name (but for use of the ordinal term) to distinguish the elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value. The term “substantially equal” may be used to refer to values that are within ±20% of one another in some embodiments, within ±10% of one another in some embodiments, within ±5% of one another in some embodiments, and yet within ±2% of one another in some embodiments.

The term “substantially” may be used to refer to values that are within ±20% of a comparative measure in some embodiments, within ±10% in some embodiments, within ±5% in some embodiments, and yet within ±2% in some embodiments. For example, a first direction that is “substantially” perpendicular to a second direction may refer to a first direction that is within ±20% of making a 90° angle with the second direction in some embodiments, within ±10% of making a 90° angle with the second direction in some embodiments, within ±5% of making a 90° angle with the second direction in some embodiments, and yet within ±2% of making a 90° angle with the second direction in some embodiments.

What is claimed is:

1. A system comprising:
a nuclear reactor for producing heat in a first medium;
a heat exchanger, thermally coupled to the nuclear reactor, for exchanging heat between the first medium and a second medium; and
a gasification reactor for receiving heat from the second medium to convert input biomass into output syngas.

2. The system of claim 1, wherein the nuclear reactor produces heat using tri-structural isotropic (TRISO) particle fuel.

3. The system of claim 1, wherein the first medium comprises helium and the second medium comprises helium, or a molten salt, or steam, or a liquid metal.

4. The system of claim 1, wherein the first medium has a temperature of up to 1600° C. and the second medium has a temperature of between 800° C. and 1300° C.

5. The system of claim 1, wherein the nuclear reactor further produces radioactive medical isotopes.

6. The system of claim 1, further comprising a Fischer-Tropsch reactor receiving the syngas for converting the syngas into one or more selected hydrocarbons using a Fischer-Tropsch process.

7. The system of claim 6, wherein one or more of the selected hydrocarbons comprises aviation fuel or carbon fiber.

8. The system of claim 6, wherein the heat used in the Fischer-Tropsch process is received primarily from the received syngas.

9. The system of claim 6, wherein a temperature of the Fischer-Tropsch process does not exceed 800° C.

10. A method comprising:
producing heat in a first medium using a nuclear reactor;
exchanging heat between the first medium and a second medium using a heat exchanger thermally coupled to the nuclear reactor; and
converting input biomass into output syngas using a gasification reactor that receives heat from the second medium.

11. The method of claim 10, wherein producing heat in the first medium comprises producing nuclear reactions inside tri-structural isotropic (TRISO) particle fuel.

12. The method of claim 10, wherein the first medium comprises helium and the second medium comprises helium, or a molten salt, or a liquid metal.

13. The method of claim 10, wherein exchanging heat comprises raising the second medium to a temperature of between 800° C. and 1300° C.

14. The method of claim 10, further comprising producing radioactive medical isotopes using the nuclear reactor.

15. The method of claim 10, further comprising converting the syngas into one or more selected hydrocarbons using a Fischer-Tropsch process in a Fischer-Tropsch reactor.

16. The method of claim 15, wherein one or more of the selected hydrocarbons comprises aviation fuel or carbon fiber.

17. The method of claim 15, wherein the heat used in the Fischer-Tropsch process is received primarily from the second medium and the received syngas.

18. The method of claim 15, wherein a temperature of the Fischer-Tropsch process does not exceed 800° C.
